(19) United States
(12) Patent Application Publication
(10) Pub. No.: US 2015/0344530 A1
(43) Pub. Date: Dec. 3, 2015

(54) Synthetic Peptides as Carriers for Conjugation with Polysaccharides

(71) Applicant: Subhash V. Kapre, Redmond, WA (US)

(72) Inventor: Subhash V. Kapre, Redmond, WA (US)

(21) Appl. No.: 14/718,315

(22) Filed: May 21, 2015

Related U.S. Application Data
(60) Provisional application No. 62/004,600, filed on May 29, 2014.

(51) Int. Cl. C07K 14/315; A61K 47/48; A61K 39/102; A61K 39/112; A61K 39/09; A61K 39/095

(52) U.S. Cl. CPC C07K 14/3156; A61K 39/092; A61K 39/095; A61K 39/102; A61K 39/0275; A61K 47/48238; A61K 2039/70

(57) ABSTRACT

The invention is directed to compositions and methods for the manufacture and administration of vaccines wherein the peptide comprises at least 28 amino acid residues of pneumococcal surface adhesion A and, in particular compositions, methods and tools for the conjugation of peptides with polysaccharides and other chemical agents in the formulation of vaccines.

### REFERENCE TO RELATED APPLICATIONS

This application claims priority to U.S. Provisional Application No. 62/004,600 of the same title and filed May 29, 2014, the entirety of which is specifically incorporated by reference.

### SEQUENCE LISTING

The instant application contains a Sequence Listing which has been submitted in ASCII format via EFS-Web and is hereby incorporated by reference in its entirety. Said ASCII copy, created on May 20, 2015, is named 30.36.007.US_SL.txt and is 7,933 bytes in size.

### BACKGROUND OF THE INVENTION

1. Field of the Invention

The invention is directed to compositions and methods for the manufacture and administration of vaccines and, in particular compositions, methods and tools for the conjugation of peptides with polysaccharides and other chemical agents in the formulation of vaccines.

2. Description of the Background

An immune response is a complex series of cell-to-cell interactions. Briefly, a foreign antigen enters the body and encounters antigen-presenting cells that process the antigen and retain fragments of the antigen on their surfaces. The antigen fragments retained on the surfaces of presenting cells are recognized by T cells that then induce B cells to proliferate and divide into antibody forming cells. These B cells then secrete antibody that is specifically reactive against the foreign antigen. Most antigens only elicit antibodies with assistance from the T cells and, hence, are known as T-dependent. T-dependent antigens include, for example, tetanus toxoid and diphtheria toxoid. Some antigens, such as polysaccharides, cannot be properly processed by antigen presenting cells and are not recognized by T cells. These antigens do not require T cell assistance to elicit antibody formation, but can activate B cells directly and, hence, are known as T-independent antigens. T-independent antigens include, for example, H. influenza type b polyribosyl-ribitol-phosphate (PRP) and pneumococcal capsular polysaccharides. There are a number of additional differences between T-independent and T-dependent antigens. For example, (i) T-dependent antigens, but not T-independent antigens, can prime an immune response so that a memory response results on secondary challenge with the same antigen; (ii) the affinity of the antibody for antigen increases with time after immunization with T-dependent, but not T-independent antigens; (iii) T-dependent antigens stimulate an immature or neonatal immune system more effectively than T-independent antigens; and (vi) T-dependent antigens usually stimulate IgM, IgG1, IgG2a, and IgE antibodies, while T-independent antigens stimulate IgM, IgG1, IgG2b, and IgG3 antibodies. T-dependent antigens stimulate primary and secondary responses, which are long-lived in both adult and in neonatal immune systems. However, sufficient protection against the pathogen often require frequent be vaccinations that include adjuvants. Also, very small proteins, such as peptides, are rarely immunogenic, even when administered with adjuvants.

T-independent antigens, such as polysaccharides, are able to stimulate immune responses in the absence of adjuvants, but cannot stimulate high level or prolonged antibody responses. They are also unable to stimulate an immature or B cell defective immune system. For T-independent antigens, it is desirable to provide protective immunity against such antigens to children, especially against capsular polysaccharides found on organisms such as H. influenza, S. pneumonia, and Neisseria meningitis. Enhancement of the immune response to T-independent antigens involves conjugating polysaccharides such as H. influenza PRP or oligosaccharide antigens to a T-dependent antigen such as tetanus or diphtheria toxoid. This allows for the recruitment of T cell help which provides enhanced immunity to a host.

Protein-polysaccharide conjugate vaccines stimulate an anti-polysaccharide antibody response in hosts who are otherwise unable to respond to the polysaccharide alone, such as infants and the immunocompromised. Conjugation of a protein and a polysaccharide also enhances the antibody response not only to the polysaccharide component, but also to the protein component. Many chemical techniques have been developed to facilitate coupling of proteins and polysaccharides. (e.g., Dick, W. E. et al., “Glyconjugates of Bacterial Carbohydrate Antigens: A Survey and Consideration of Design and Preparation Factors,” Conjugate Vaccines. Eds. Cruse, et al., p. 48, 1989). Homofunctional and heterofunctional vinylsulfone reagents have been used to activate polysaccharides. The activated polysaccharides are reacted with a protein, peptide, or hapten, under appropriate reaction conditions, to produce the conjugate. Another method for producing conjugate vaccines comprises mixing a uronium salt reagent with a soluble first moiety, such as a polysaccharide or carbohydrate, and combining therewith a second moiety, such as a protein, peptide, or carbohydrate, to form the conjugate vaccine.

Carbohydrates can also be activated before conjugation with chemicals such as cyanogen bromide (CNBr). CNBr-activation involves reacting CNBr with the carbohydrate of interest at a high pH, typically from about 10 to 12. At the higher pH range, cyanate esters are formed with the hydroxyl groups of the carbohydrate. These esters are reacted with a bifunctional reagent, such as a diamine or a dihydrazide. These resulting derivatized carbohydrates may then be conjugated via the bifunctional group. In certain cases, the cyanate esters may also be directly reacted to protein. CNBr itself is highly unstable and spontaneously hydrolyzes at high pH, which can reduce overall yield. A high pH is generally necessary to ionize the hydroxyl group because the reaction requires the nucleophilic attack of the hydroxyl ion on the cyanate ion. However, CNBr produces many side reactions, some of which add neo-antigens to the polysaccharides. Wilcheck, M. et al., Affinity Chromatography. Meth. Enzymol., 104: 3-55 (1984). Carbohydrates or moieties such as Hib, PRP and capsular polysaccharides from and pneumococcal type 6 and Neisseria meningitis A can be hydrolyzed and damaged by the high pH necessary to perform the cyanogen bromide activation. Also, the cyanate ester formed is generally unstable at high pH and rapidly hydrolyzes. This can reduce the yield of derivatized carbohydrate and, hence, the overall yield of carbohydrate conjugated to protein. Nonproductive side reactions, such as those producing carbamates and linear imidocarbonates, are promoted by the high pH. This effect is described in Kohn et al., Anal. Biochem, 115: 375 (1981).

1-cyano-4-(dimethylamino)-pyridinium tetrafluoroborate (CDAP) has been used in aqueous media to activate polysaccharides. These activated polysaccharides may be directly or indirectly coupled to proteins. CDAP chemistry is described in, for example, U.S. Pat. No. 5,849,301 and in Lees, et al., “Activation of Soluble Polysaccharides with 1-Cyano-4-Dimethylamino Pyridinium Tetrafluoroborate For Use in Protein-Polysaccharide Conjugate Vaccines and Immunological Reagents,” Vaccine, 14(3): 190 (1996).

Protein-polysaccharide conjugate vaccines may also be formed via reductive amination. In this method, aldehydes on the polysaccharide are reacted with amines on the protein to form a reversible Schiff base. The Schiff base is subsequently reduced to form a stable linkage between the amine and the aldehyde. Formation of a Schiff base can be impeded by the size of the components (i.e., the polysaccharide and protein), as the components need to be in close proximity to react. Often to overcome this problem the polysaccharide is broken down into oligosaccharides prior to chemical coupling. Dimethylsulfoxide (DMSO) promotes the formation of the Schiff base, but organic solvents such as DMSO can damage the protein. Sometimes a multistep protocol is used, in which a spacer group (e.g., hexane diamine or adipic dihydrazide) is added to the polysaccharide via reductive amination, and this spacer is subsequently ligated to the protein. A high concentration of the spacer helps to force the reaction and increase yield. Elevated temperatures and prolonged reaction times are also used to promote the reaction. Depending of the chemical structures utilized, either can also be detrimental to the protein and/or the polysaccharide. Furthermore, as amines must be deprotonated to react with aldehydes, the Schiff base formation usually requires the use of alkaline solutions. Furthermore, the reductive step, which usually involves cyanoborohydride or pyridine-boranes, can be inefficient and deleterious to the protein. Of course, these reagents are hazardous to work with in large quantities. Reductive amination is highly random with regard to the linkage sites between the protein and the polysaccharide. Accordingly, there remains a need in the art for an efficient and effective process for preparing conjugate vaccines.

### SUMMARY OF THE INVENTION

The present invention overcomes the problems and disadvantages, associated with current strategies and designs and provides new composition, tools and methods for the development and manufacture of conjugate and vaccines.

One embodiment of the invention is directed to compositions comprising: a polysaccharide conjugated to a peptide wherein the peptide comprises at least 28 amino acid residues of pneumococcal surface adhesion A (SEQ ID NO 4). Preferably the peptide comprises P4 peptide (SEQ. ID NO. 1) or a P4 peptide containing conservative amino acid variations. Preferably the conjugate contains one or more additional cysteine amino acid residues, to which are the peptides via one end of the one or more cysteine amino acid residues and the polysaccharide is covalently linked to the other end of the one or more cysteine amino acid residues. Preferably the peptide and the polysaccharide are covalently linked or the peptide is covalently linked to an amino, hydroxyl or carboxyl residues of the polysaccharide. Also preferably, the peptide and the polysaccharide may be covalently linked through a linker and the linker is adipic acid, dihydrazide, hydrazine or a water-soluble N-hydroxy succinimide ester. Preferably, the conjugate is prepared by carbidiimide chemical processing which typically requires a coupling agent such as, for example, EDC, DIC and/or DCC. Preferably the peptide contains a sulfhydryl group and the polysaccharide contains a maleimide or halo acetyl group, or the polysaccharide contains a sulfhydryl group and the peptide contains a maleimide or halo acetyl group. Preferably the polysaccharide is derived from a bacterial polysaccharide such as, for example, a serotype of Streptococcus pneumoniae, Neisseria meningitis, Haemophilus Influenzae, Staphylococcus aureus, or Salmonella typhi, Salmonella paratyphi or non-typeable Salmonella. Preferably the polysaccharide is derivitized and linked to the peptide through an aldehyde, an amino group or a carboxyl group and the polysaccharide is composed of at least 4-10000, 4-3000, 4-500 repeating units. Also preferably, the conjugate comprises a molar ratio of peptide to polysaccharide of 1:1, 1:2, 1:3, 1:5, 1:20, 1:30 or 1:50.

Another embodiment of the invention is directed to pharmaceutical compositions comprising the conjugate of the invention. Preferably the pharmaceutical composition further comprises a pharmaceutically acceptable diluent and/or an adjuvant, and the pharmaceutical composition is a vaccine. Preferably the polysaccharide is derived from one or more subtypes of Streptococcus pneumonia, and also preferably, the serotype is selected from the group consisting of 1, 2, 3, 4, 5, 6B, 7F, 8, 9N, 9V, 10A, 11A, 12F, 14, 15B, 17F, 18C, 19F, 19A, 20, 22F, 23F and 33F.

Another embodiment of the invention is directed to processes for preventing or treating a disorder comprising administering to a patient an immunologically effective amount of a vaccine comprised of the conjugate of the invention. Preferably the vaccine is a multi-variant vaccine.

Another embodiment of the invention comprising: a polysaccharide conjugated to a peptide wherein the peptide comprises one or more of SEQ ID NOs 1-8 and/or combinations thereof.

Other embodiments and advantages of the invention are set forth in part in the description, which follows, and in part, may be obvious from this description, or may be learned from the practice of the invention.

### DESCRIPTION OF THE FIGURES

FIG. 1 Description of the chemical process of carbodiimide chemistry.

### DESCRIPTION OF THE INVENTION

Most vaccines are marketed and licensed as proteins-polysaccharide conjugates. For example, Pneumococcal conjugate vaccines, Meningococcal conjugate vaccines, Haemophilus influenza type b conjugate vaccines, and Salmonella typhi conjugate vaccines each contain a polysaccharide isolated from bacterial source and a carrier protein which is Tetanus/Diphtheria toxoid, CRM197 or Protein D. Immunogenic peptides or helper peptides contain epitopes recognized by T helper cells that induce an immune response to a particular pathogen.

The invention is directed to a polysaccharide-peptide conjugate wherein the polysaccharide is advantageously immunogenic, and peptide moiety, which may contain additional cysteine residues, is covalently attached, either randomly or selectively, along the polysaccharide chain directly or through a spacer-linker. It has been surprisingly discovered that a conjugate vaccine composed of a polysaccharide-peptide linkage wherein the peptide preferably comprises P4 peptide (SEQ. ID NO. 1) (a 28 amino acid sequence of PsaA) or another peptide that comprises at least 28 amino acid residues of the pneumococcal surface adhesion A (PsaA) (SEQ ID NO 2) provides an effective vaccine. Preferably the peptide is P4 peptide with the amino acid sequence of SEQ ID NO: 1. Although P4 peptides and other peptides are preferably used as is, P4 and other PsaA peptides may also be modified prior to or contemporaneously with the development of the conjugate molecule. Preferred modifications include, but are not limited to one or more modifications of one or more side chains, one or more modifications to a carboxyl, amino or hydroxy group. The composition comprising a conjugate of polysaccharide plus P4 peptide generates opsonic antibodies or fragments that specifically bind to an antigen present on the surface of the pathogen and enhances opsonophagocytosis.

One embodiment of the invention is directed to a composition comprising a polysaccharide conjugated to a peptide. Preferably the polysaccharide and the peptide are covalently linked though an amino, hydroxyl or carboxyl residues at the terminal or internal regions of the polysaccharide. Also preferably the peptide and the polysaccharide may be covalently linked through a linker. Preferred linkers include, for example, adipic acid, dihydrazide, hydrazine, a water-soluble N-hydroxy succinimide ester and combinations thereof.

The peptide preferably comprises a series of residues derived or obtained from pneumococcal surface adhesion A (PsaA) (SEQ ID NO 4), or a sequence containing conservative amino acid modifications of PsaA. Preferably the series comprises at least 15 amino acids derived from PsaA, more preferable at least 20 amino acids, more preferably at least 25 amino acids, and more preferable at least 28 or P4 peptide (SEQ ID NO 1), and more preferably at least 30 amino acids or more. The peptide may be modified with the addition of one or more cysteine residues on either the carboxyl (C) or amino (N) terminal positions of the terminal regions or of using the natural PsaA protein sequence (e.g., P4 peptide). Also preferably, the peptide contains a sulfhydryl group and the polysaccharide contains a maleimide or halo acetyl group. Preferably the polysaccharide is derived or obtained from a pathogen such as, for example, a bacterial, fungal, viral or parasitic organism or microorganism. The conjugate of the polysaccharide is preferably immunogenic for the polysaccharide and, when administered to a mammal, generates a humoral response (e.g., antibody, compliment and compliment cascade-related proteins) and/or cellular response (e.g., T cells, natural killer cells, macrophages and B cells) of the immune system. Also preferably, that immune response provides protection from and/or treatment of an infection due to the particular pathogen or other disease or disorder. In addition, the polysaccharide may be associated with a cancer, such as, for example, prostate, pancreatic thyroid, breast, bladder, endometrial, lung, colon and rectal, and head and neck cancers, melanoma, leukemia, lymphoma and metastatic disease. Also preferably, the polysaccharide contains a sulfhydryl group. Preferably the bacterial polysaccharide is a serotype of Streptococcus pneumoniae, Neisseria meningitis, Haemophilus Influenzae, Staphylococcus aureus, or Salmonella typhi, paratyphi or non-typeable salmonella. Also preferably, the polysaccharide is derivitized and linked to the peptide through an aldehyde, an amino group or a carboxyl group, and is composed of at least four 10,000 KD, four 3,000 KD, and/or four 500 KD repeating units or comprises a molar ratio of peptide to polysaccharide of 1:1, 1:2, 1:3, 1:5, 1:20, 1:30 or 1:50.

Preferably, the conjugate contains one or more additional cysteine amino acid residues that may be linked to terminal or internal chemical moieties of the conjugate. Alternatively, or in addition, the cysteine may serve as a linker between the peptide and the polysaccharide such as, for example, wherein the peptide is covalently linked to one end of one or more cysteine amino acid residues and the polysaccharide is covalently linked to the other end of the one or more cysteine amino acid residues. Preferably, the conjugate is formed with carbidiimide chemical processing that includes, for example a couple agent. Preferred coupling agents include, for example, EDC, DIC and DCC.

Another embodiment of the invention is directed to a composition comprising the conjugate of claim 1. The composition may be a pharmaceutical composition and may include a pharmaceutically acceptable diluent and/or an adjuvant. Preferably the pharmaceutical composition is a vaccine. Preferably the vaccine comprises a polysaccharide derived from one or more subtypes of Streptococcus pneumonia. Serotype include, but are not limited to 1, 2, 3, 4, 5, 6B, 7F, 8, 9N, 9V, 10A, 11A, 12F, 14, 15B, 17F, 18C, 19F, 19A, 20, 22F, 23F and 33F.

Another embodiment of the invention is directed to a process for preventing or treating a disorder comprising administering to a patient an immunologically effective amount of a vaccine comprised of the conjugate of the invention. The vaccine of the invention is preferably a multi-variant vaccine. Also preferably, the conjugate is formed by a carbodiimide chemical process and the carbodiimide or methanediimine is a functional group consisting of the formula RN═C═NR. Carbodiimides are formed by dehydration of ureas or thioureas (forming carbon dioxide), or by treating organic isocyanates with a catalyst, such as preferably phosphine oxides. According to the preferred process, compounds containing the carbodiimide functionality are dehydration agents used to activate carboxylic acids towards amide or ester formation. N-hydroxybenzotriazole or N-hydroxysuccinimide can be included as additives to increase yields and decrease side reactions. Preferably, the coupling agent is 1-Ethyl-3-(3-dimethylaminopropyl)carbodiimide (EDC), N,N′-dicyclohexylcarbodiimide (DCC), or N,N′-diisopropylcarbodiimide (DIC).

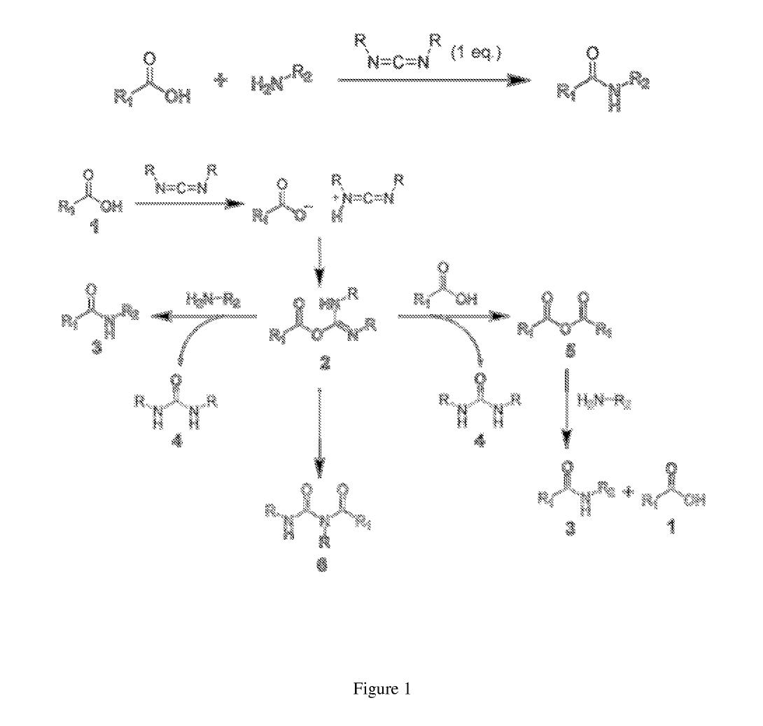

Carbodiimide chemistry involves forming amide from carbodiimide, but there may be side reactions complicating the result (see U.S. Pat. No. 2,853,518). As depicted in FIG. 1, acid 1 reacts with carbodiimide to produce the key intermediate, O-acylisourea 2, which can be viewed as a carboxylic ester with an activated leaving group. The O-acylisourea reacts with amines to produce amide 3 and urea 4. The side reaction of the O-acylisourea 2 produces both the desired product, but also possible undesired products. The O-acylisourea 2 can react with an additional carboxylic acid 1 to give an acid anhydride 5, which can react further to give the desired amide 3. The main undesired reaction pathway involves the rearrangement of the O-acylisourea 2 to the stable N-acylurea 6. The use of solvents with low-dielectric constants such as dichloromethane or chloroform can minimize this side reaction.

DCC is often used for amide and ester formation, especially for solid-phase peptide synthesis. The widespread use of DCC is mainly been due to its high yielding amide coupling reactions and the fact that it is quite inexpensive. DIC is similar to DCC and equally useful. EDC is a water soluble carbodiimide that can be obtained as the hydrochloride. It is useful in the 4.0-6.0 pH range as a carboxyl activating agent for the coupling of primary amines to yield amide bonds. Additionally, EDC can also be used to activate phosphate groups to form phosphomono-esters and phosphodiesters. Common uses for this carbodiimide include peptide synthesis, protein crosslinking to nucleic acids, but also in the preparation of immunoconjugates. EDC is often used in combination with N-hydroxysuccinimide (NHS) or sulfo-NHS to increase coupling efficiency by creating a more reactive amine-product. Carbodiimides can also react with amines to form guanidines. Additional chemical procedures for coupling a peptide with a polysaccharide are disclosed and described in U.S. Patent Application Publication Nos. 2010/0016546, 2012/0288533 and 2013/0195893, International Application Publication Nos. WO 97/26784, WO 98031393, WO 98/31393, and WO 2014/053521 U.S. Pat. Nos. 5,679,582, 6,472,506 and 7,691,641, Canadian Patent Application No. 2,246,760, and European Patent No. 0959905, all of which are specifically and entirely incorporated by reference.

The following examples illustrate embodiments of the invention, but should not be viewed as limiting the scope of the invention.

### EXAMPLES

### Example 1

### Sequence Information

SEQ ID NO 1:
LFVESSVKRR PMKTVSQDTN IPIYAQIF (P4 Peptide)
SEQ ID NO 2:
EKLRQTKVPSL FVESSVKRRP MKTVSQDTNI PIYAQIFTDSI
SEQ ID NO 3:
EKLRQTKVPSL FVESSVKRRP MKTVSQDTNI PIYAQIFTDSI
LFVESSVKRR PMKTVSQDTN IPIYAQIF
SEQ ID NO 4:
MKKLGTLLVL FLSAIILVAC ASGKKDTTSG QKLKVVATNS
IIADITKNIA GDKIDLHSIVPIGQDPHEYE PLPEDVKKTS
EADLIFYNGI NEITGGNAWF TKLVENAKKT
ENKDYFAVSDGVDVIYLEGQ NEKGKEDPHA WLNLENGIIF
AKNIAKQLSA KDPNNKEFYE KNLKEYTDKLDKLDKESKDK
FNKIPAEKKL IVTSEGAFKY FSKAYGVPSA YIWEINTEEE
GTPEQIKTLVEK EGDSYYSMMKYNLDKIAGGLAK
SEQ ID NO 5:
MKKLGTLLVL FLSAIILVAC ASGKKDTTSG QKLKVVATNS
IIADITKNIA GDKIDLHSIVPIGQDPHEYE PLPEDVKKTS
EADLIFYNGI NEITGGNAWF TKLVENAKKT
ENKDYFAVSDGVDVIYLEGQ NEKGKEDPHA WLNLENGIIF
AKNIAKQLSA KDPNNKEFYE KNLKEYTDKLDKLDKESKDK
FNKIPAEKKL IVTSEGAFKY FSKAYGVPSA YIWEINTEEE
GTPEQIKTLVEK LRQTKVPS LFVESSVKRRPMKTVSQDTNIPIYAQIF
TDSIAEQGK EGDSYYSMMKYNLDKIAGGLAK
SEQ ID NO 6:
LRQTKVPS
SEQ ID NO 7:
LFVESSVKRRPMKTVSQDTNIPIYAQIF
SEQ ID NO 8:
TDSIAEQGK

Other embodiments and uses of the invention will be apparent to those skilled in the art from consideration of the specification and practice of the invention disclosed herein. All references cited herein, including all publications, U.S. and foreign patents and patent applications, are specifically and entirely incorporated by reference. The term comprising, where ever used, is intended to include the terms consisting and consisting essentially of. Furthermore, the terms comprising, including, and containing are not intended to be limiting. It is intended that the specification and examples be considered exemplary only with the true scope and spirit of the invention indicated by the following claims.

1. A composition comprising: a peptide comprising at least 28 amino acid residues of pneumococcal surface adhesion A (SEQ ID NO 4) conjugated to a polysaccharide.

2. The composition of claim 1, wherein the peptide comprises P4 peptide (SEQ. ID NO. 1).

3. The composition of claim 1, wherein the conjugate contains one or more additional cysteine amino acid residues.

4. The composition of claim 3, where the peptide is covalently linked to one end of the one or more cysteine amino acid residues and the polysaccharide is covalently linked to the other end of the one or more cysteine amino acid residues.

5. The composition of claim 1, wherein the peptide and the polysaccharide are covalently linked.

6. The composition of claim 5, where peptide is covalently linked to an amino, hydroxyl or carboxyl residues of the polysaccharide.

7. The composition of claim 1, where the peptide and the polysaccharide are covalently linked through a linker and the linker is adipic acid, dihydrazide, hydrazine or a water-soluble N-hydroxy succinimide ester.

8. The composition of claim 1, which is conjugated by carbidiimide chemical processing.

9. The composition of claim 1, wherein the carbidiimide chemical process comprises a coupling agent.

10. The composition of claim 9, wherein the coupling reagent is EDC, DIC or DCC.

11. The composition of claim 1, wherein the peptide contains a sulfhydryl group and the polysaccharide contains a maleimide or halo acetyl group.

12. The composition of claim 1, wherein the polysaccharide contains a sulfhydryl group and the peptide contains a maleimide or halo acetyl group.

13. The composition of claim 1, where the polysaccharide is derived from a bacterial polysaccharide.

14. The composition of claim 1, where the bacterial polysaccharide is a serotype of Streptococcus pneumoniae, Neisseria meningitis, Haemophilus Influenzae, Staphylococcus aureus, or Salmonella typhi, Salmonella paratyphi or non-typeable Salmonella.

15. The composition of claim 1, where the polysaccharide is derivitized and linked to the peptide through an aldehyde, an amino group or a carboxyl group.

16. A conjugate according to the claim 1, wherein the polysaccharide is composed of at least 4-10000, 4-3000, 4-500 repeating units.

17. The composition of claim 1, where the conjugate comprises a molar ratio of polysaccharide to peptide of: 1:1, 1:2, 1:3, 1:5, 1:20, 1:30 or 1:50.

18. A pharmaceutical composition comprising the conjugate of claim 1.

19. The pharmaceutical composition of claim 18, further comprising a pharmaceutically acceptable formulation.

20. The pharmaceutical composition of claim 18, further comprising an adjuvant.

21. The pharmaceutical composition of claim 18, which is a vaccine.

22. The pharmaceutical composition of claim 21, wherein the polysaccharide is derived from one or more subtypes or serotypes of Streptococcus pneumonia.

23. The pharmaceutical composition of claim 22, wherein the one or more subtypes or serotype are one or more of 1, 2, 3, 4, 5, 6B, 7F, 8, 9N, 9V, 10A, 11A, 12F, 14, 15B, 17F, 18C, 19F, 19A, 20, 22F, 23F, 33F and combinations thereof.

24. The pharmaceutical composition of claim 21, wherein the polysaccharide is derived from one or more subtypes or serotypes of Niesseria meningitidis.

25. The pharmaceutical composition of claim 21, wherein the one or more subtypes or are selected from A,C,Y,W135 and combinations thereof.

26. The pharmaceutical composition of claim 21, wherein the polysaccharide is derived from Haemophilus Influenzae serotypes.

27. The pharmaceutical composition of claim 21, wherein the polysaccharide is derived from Salmonella Typhi.

28. The pharmaceutical composition of claim 21, wherein the polysaccharide is derived from a microorganism.

29. A process for preventing or treating a disorder comprising administering to a patient an immunologically effective amount of a vaccine comprised of the conjugate of claim 1.

30. The process of claim 29, wherein the vaccine is a multi-variant vaccine.

31. A composition comprising: a polysaccharide conjugated to a peptide wherein the peptide comprises the sequence of one or more of the sequences of SEQ ID NOs 1-8 and/or combinations thereof.


SEQUENCE LISTING (8 sequences)

SEQ ID NO: 1 - protein, 28 aa, Artificial Sequence
  Description of Artificial Sequence Synthetic peptide
LFVESSVKRRPMKTVSQDTNIPIYAQIF

SEQ ID NO: 2 - protein, 42 aa, Artificial Sequence
  Description of Artificial Sequence Synthetic polypeptide
EKLRQTKVPSLFVESSVKRRPMKTVSQDTNIPIYAQIFTDSI

SEQ ID NO: 3 - protein, 70 aa, Artificial Sequence
  Description of Artificial Sequence Synthetic polypeptide
EKLRQTKVPSLFVESSVKRRPMKTVSQDTNIPIYAQIFTDSILFVESSVKRRPMKTVSQDTNIPIYAQIF

SEQ ID NO: 4 - protein, 264 aa, Artificial Sequence
  Description of Artificial Sequence Synthetic polypeptide
MKKLGTLLVLFLSAIILVACASGKKDTTSGQKLKVVATNSIIADITKNIAGDKIDLHSIVPIGQDPHEYEPLPEDVKKTSEADLIFYNGINEITGGNAWFTKLVENAKKTENKDYFAVSDGVDVIYLEGQNEKGKEDPHAWLNLENGIIFAKNIAKQLSAKDPNNKEFYEKNLKEYTDKLDKLDKESKDKFNKIPAEKKLIVTSEGAFKYFSKAYGVPSAYIWEINTEEEGTPEQIKTLVEKEGDSYYSMMKYNLDKIAGGLAK

SEQ ID NO: 5 - protein, 309 aa, Artificial Sequence
  Description of Artificial Sequence Synthetic polypeptide
MKKLGTLLVLFLSAIILVACASGKKDTTSGQKLKVVATNSIIADITKNIAGDKIDLHSIVPIGQDPHEYEPLPEDVKKTSEADLIFYNGINEITGGNAWFTKLVENAKKTENKDYFAVSDGVDVIYLEGQNEKGKEDPHAWLNLENGIIFAKNIAKQLSAKDPNNKEFYEKNLKEYTDKLDKLDKESKDKFNKIPAEKKLIVTSEGAFKYFSKAYGVPSAYIWEINTEEEGTPEQIKTLVEKLRQTKVPSLFVESSVKRRPMKTVSQDTNIPIYAQIFTDSIAEQGKEGDSYYSMMKYNLDKIAGGLAK

SEQ ID NO: 6 - protein, 8 aa, Artificial Sequence
  Description of Artificial Sequence Synthetic peptide
LRQTKVPS

SEQ ID NO: 7 - protein, 28 aa, Artificial Sequence
  Description of Artificial Sequence Synthetic peptide
LFVESSVKRRPMKTVSQDTNIPIYAQIF

SEQ ID NO: 8 - protein, 9 aa, Artificial Sequence
  Description of Artificial Sequence Synthetic peptide
TDSIAEQGK